(19) United States
(12) Patent Application Publication
(10) Pub. No.: US 2010/0311027 A1
(43) Pub. Date: Dec. 9, 2010

(54) METHOD FOR ENCOURAGING ORAL HYGIENE

(76) Inventor: Robert T. Postal, Glen Cove, NY (US)

Correspondence Address: HANDAL & MOROFSKY, LLC, 501 KINGS HIGHWAY EAST, Fairfield, CT, 06825-4867, US

(21) Appl. No.: 12/480,393

(22) Filed: Jun. 8, 2009

(51) Int. Cl. G09B 19/00; G09B 23/28; A61B 19/00; B65D 85/00

(52) U.S. Cl. 434/263; 206/63.5; 206/459.1; 206/459.5

(57) ABSTRACT

A party favor comprising a photo, an oral hygiene appliance and an instruction card tied together by curling ribbon is distributed at, for example, a wedding to promote oral hygiene and social interaction.

### TECHNICAL FIELD

The present invention constitutes a party favor intended for distribution, for example at a wedding. The object of this device is to promote oral hygiene and harness the power of a social gathering to achieve this objective. The device is designed in such a matter that it is socially acceptable to use it in public and this promotes the use of the device by people at the wedding party.

### CROSS REFERENCE TO RELATED APPLICATIONS

(Not applicable)

### STATEMENT REGARDING FEDERALLY SPONSORED RESEARCH OR DEVELOPMENT

(Not applicable)

### BACKGROUND OF THE INVENTION

The maintenance of oral hygiene is a critical importance not only to maintaining healthy teeth and gums, but also general overall health. Poor oral health increases the likelihood of many conditions, including cardiac disease. It is an object of the invention to promote the performance of oral hygiene tasks.

### SUMMARY OF THE INVENTION

In accordance with the invention, oral hygiene procedures, including procedures which are largely cosmetic, such as tooth whitening, are encouraged. Moreover, procedures such as tooth whitening are relatively attractive to certain individuals, and devices for performing the same may be combined with oral health promoting features. For example, tooth whiteners can be combined with fluoride treatment, anti-plaque treatments and gum disease preventative agents such as baking soda and peroxide.

In accordance with the invention the promotion of tooth whitening procedures also has the effect of improving social interaction by the removal of negative signals, such as colored teeth, and implementation of an attraction getting toothy-white smile.

The inventive item is a tooth whitening appliance or device. It comprises a body with a reservoir of tooth whitening material. The tooth whitening material is of conventional composition and is fed to the output of the device by a wick which terminates in an application brush comprising bristles through which, for example, tooth whitening material is fed. The tooth whitening material may be of a conventional formulation, for example, a gel tooth whitening material, or it may incorporate additional components designed to promote oral hygiene and dental health.

Thus, the material has a consistency similar to tooth paste. In accordance with a preferred embodiment of the invention, a screw is used to advance the material into the brush. The device has a cap to prevent leakage or other messy or unsanitary conditions.

In accordance with the invention, the inventive device is given a physical configuration resembling a makeup applicator, such as a lipstick or mascara applicator. In accordance with a preferred embodiment, the device has a silver mirror finish a color which naturally complements the glitzy accoutrements of a wedding and harmoniously blends with the white of a bride's wedding gown. In accordance with the inventive method, this supports acceptability of public use of the inventive device, resulting in the generation of acceptance and peer pressure to encourage persons at the wedding celebration to begin using the device whether it be a tooth whitener or other oral hygiene device. To the extent that the tooth whitening device includes other materials which promote dental hygiene, the increase in attractiveness provided by the tooth whitener, substantially increases the likelihood that the inventive device will, in accordance with the inventive method, continue to be used.

Generally, the inventive device is a three piece wedding favor comprising a tooth whitener, a frameable photo and an instruction card (which may be made in a variety of colors and styles) which describes the whitening pen as the advantageous cosmetic or other oral hygiene device it is. At the same time, it provides simple instructions for use. For example, in the case of tooth whitener, application and allowing the material to dry is described.

The inventive favor also includes the applicator device with, for example, an oral hygiene material such as tooth whitener.

Further included is a picture of the bride and groom taken either prior to the wedding or at the wedding. If the picture is taken at the wedding, it can be printed on various different photo papers or even on a refrigerator magnet. This would be an alternative to use of a picture provided by the bride and groom. The picture or the refrigerator magnet picture or other item is then affixed to the instruction card with a glue dot making this a suitable for framing photo that is easily removed from the instruction card. Silicone adhesive may be used as a suitable glue in accordance with the present invention. However, any other glue which may be cleanly removed or which will leave a minimal and non-objectionable residue is appropriate.

All of the above elements are tied together with curling ribbon, for example, in white or in the bride's coordinated colors. Ribbon of the type which curls when pulled between a blunt knife edge bearing against a finger, in the conventional manner, is used.

The inventive device may be customized in a number of different ways ranging from different color ribbons to the type and style of photo to the different styles of instruction card.

### BRIEF DESCRIPTION THE DRAWINGS

The above and other objects and advantages of the inventive method and device will become apparent from the following description taken in conjunction with the drawings, in which:

FIG. 1 is a top plan view of the inventive wedding favor incorporating an oral hygiene device;

FIG. 2 is a top plan view of a component of the inventive device comprising a photograph;

FIG. 3 is a plan view of the instruction sheet of the inventive wedding favor;

FIG. 4 is a top plan view of the inventive wedding favor incorporating an oral hygiene device with the binding ribbon removed to illustrate more clearly the components;

FIG. 5 is a bottom plan view of the inventive wedding favor incorporating an oral hygiene device with the binding ribbon removed to expose more clearly the components;

FIG. 6 is a side view of the inventive wedding favor incorporating an oral hygiene device along line 6-6 of FIG. 4 with the binding ribbon removed to illustrate more clearly the components;

FIG. 7 is a side view of the inventive applicator device separate from the inventive wedding favor; and

FIG. 8 is an exploded side view of the inventive applicator device separate from the inventive wedding favor.

### DETAILED DESCRIPTION OF THE PREFERRED EMBODIMENTS

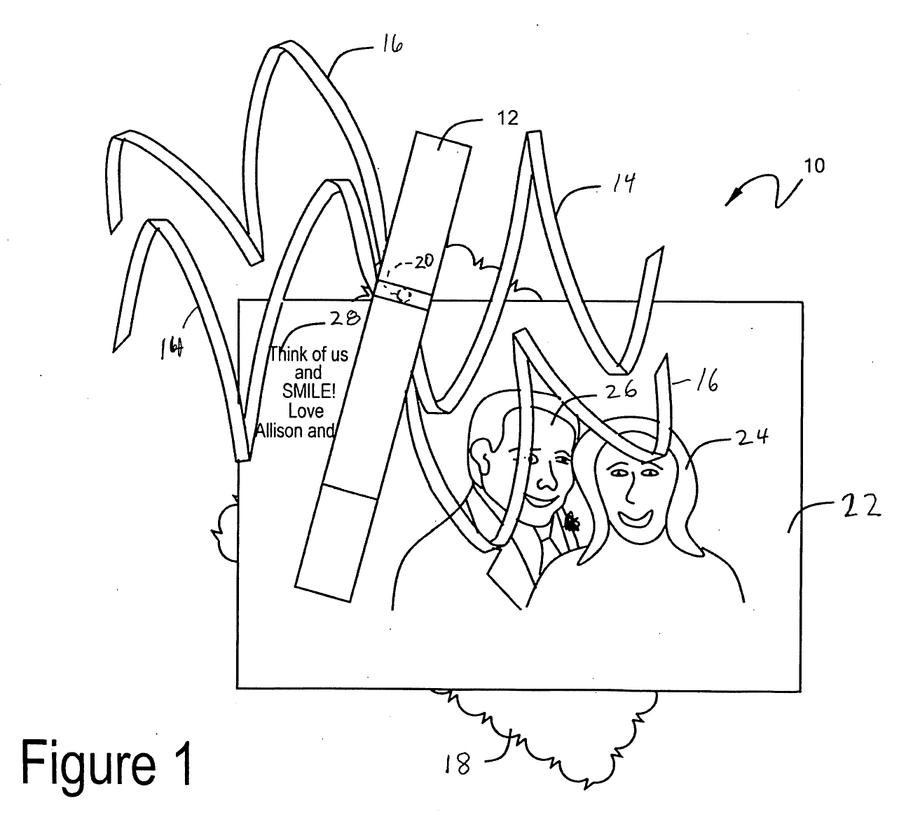

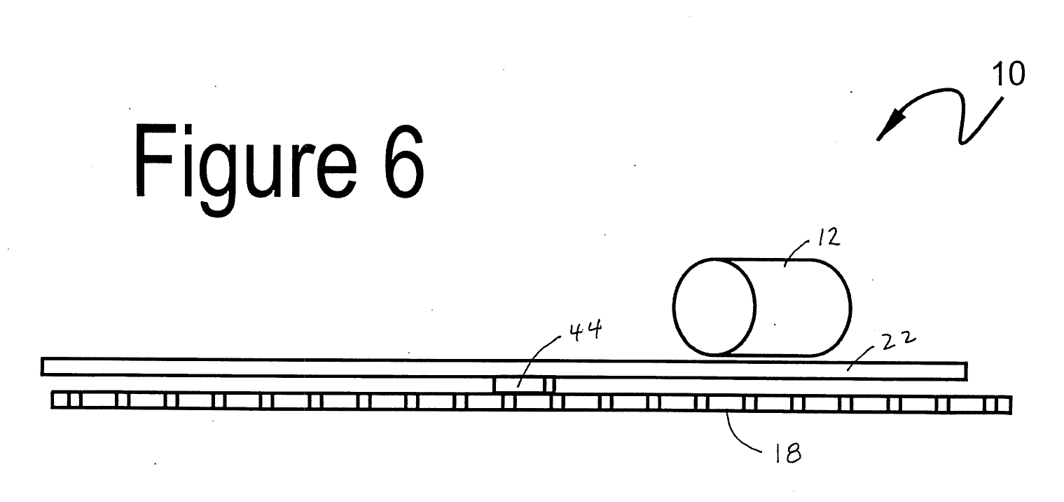

Referring to FIGS. 1-6, the inventive wedding favor 10 comprises applicator 12 which is designed to apply an oral hygiene material to the teeth. Suitable oral hygiene materials include whiteners, fluoride, anti-plaque compositions, materials for the treatment of gum disease, material for the treatment of periodontal disease, breath fresheners, and so forth.

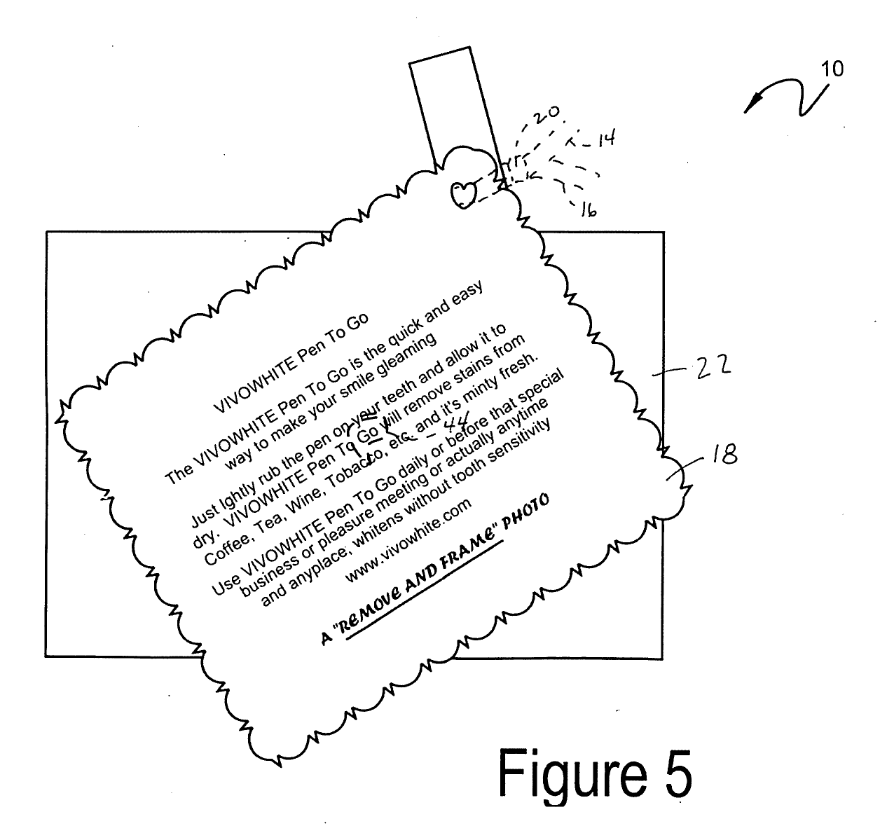

Applicator 10 is secured by ribbons 14 and 16 to message card 18. Ribbons 14 and 16 secure applicator 12 to instruction or message card 18 by being tied with a knot 20 as illustrated in phantom lines in FIG. 5.

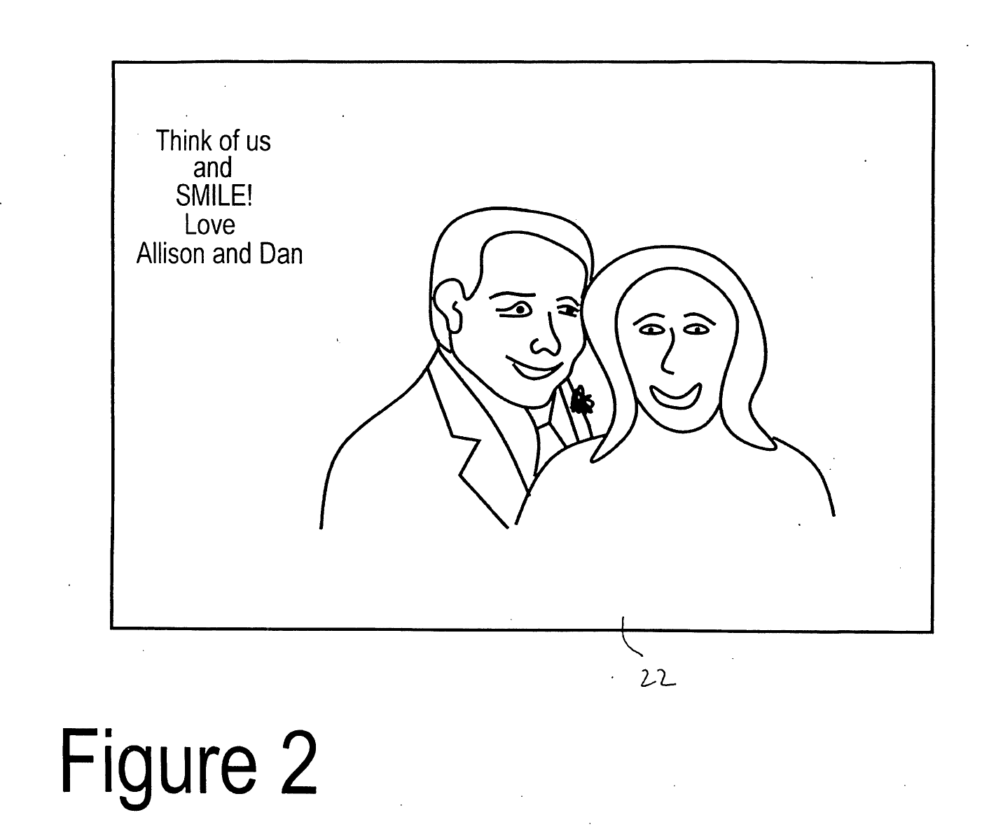

Inventive favor 10 further comprises a photograph 22 bearing, for example, images of a bride 24 and a groom 26. Photograph 22 also has a greeting 28, in the instant example being described, “Think of us and SMILE! Love Alison and Dan”. See FIG. 2.

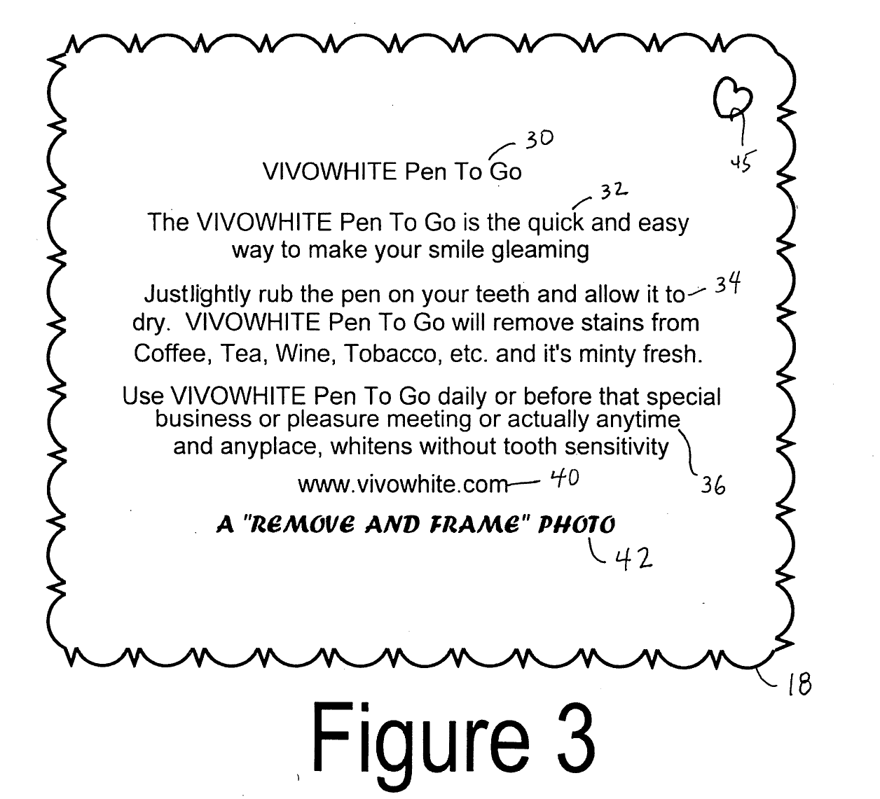

As shown in FIG. 3 the instruction card describes the product and its use with information which is printed on card 18 using a conventional process. Card 18 may be made of paper, plastic or any other suitable material. More particularly, it includes a product identification 30, a product description 32 and a product use direction 34. A suggested application is described by printed information 36.

In accordance with the preferred embodiments of the invention, the recipient is provided with the website address of the maker by printed element 40. This enhances the effectiveness of the present invention by providing a convenient mechanism for users to be informed of a source for continuing supply.

In accordance with the invention, it is contemplated that photograph 22 may be removed and framed. This information is indicated by alphanumeric imprinting 42, as illustrated in FIG. 3. Alphanumeric imprinting 42 is printed in an easily visible and attention demanding type font, thus informing the user of an attractive feature of the invention.

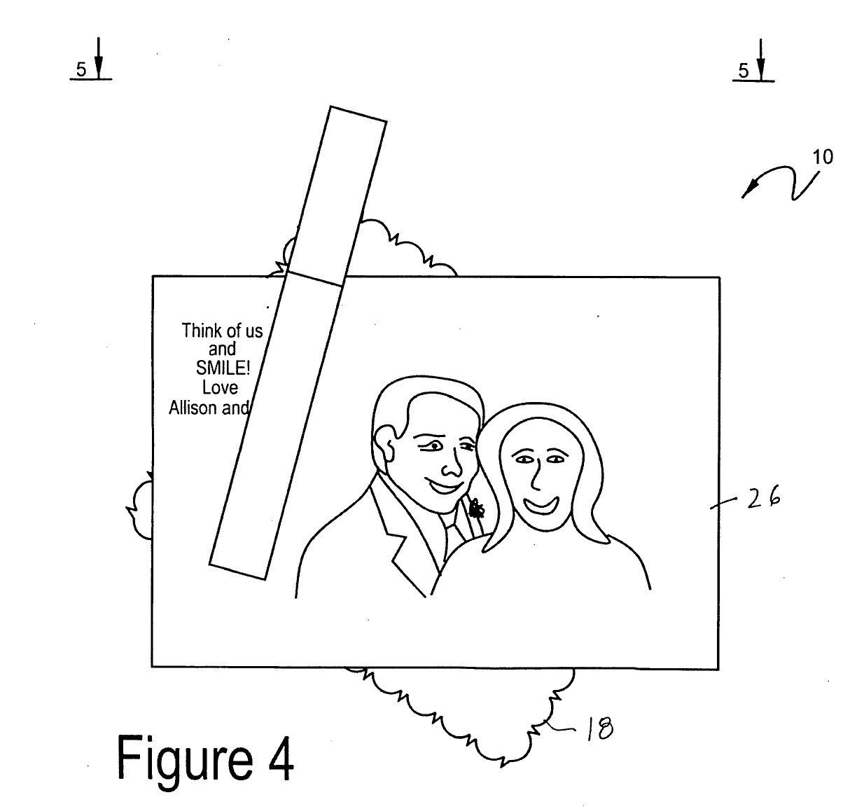

The position of the various constituent parts of the invention, without the ribbon is illustrated in FIGS. 4 and 5. In accordance with the invention it is desired that photograph 22 not be damaged during the disassembly of the inventive wedding favor 10. Accordingly, a small spot of glue 44 is used to secure photograph 22 to card 18.

Card 18 has a hole 45 through which ribbons 14 and 16 are passed before being bound around applicator 12 and tied into knot 20.

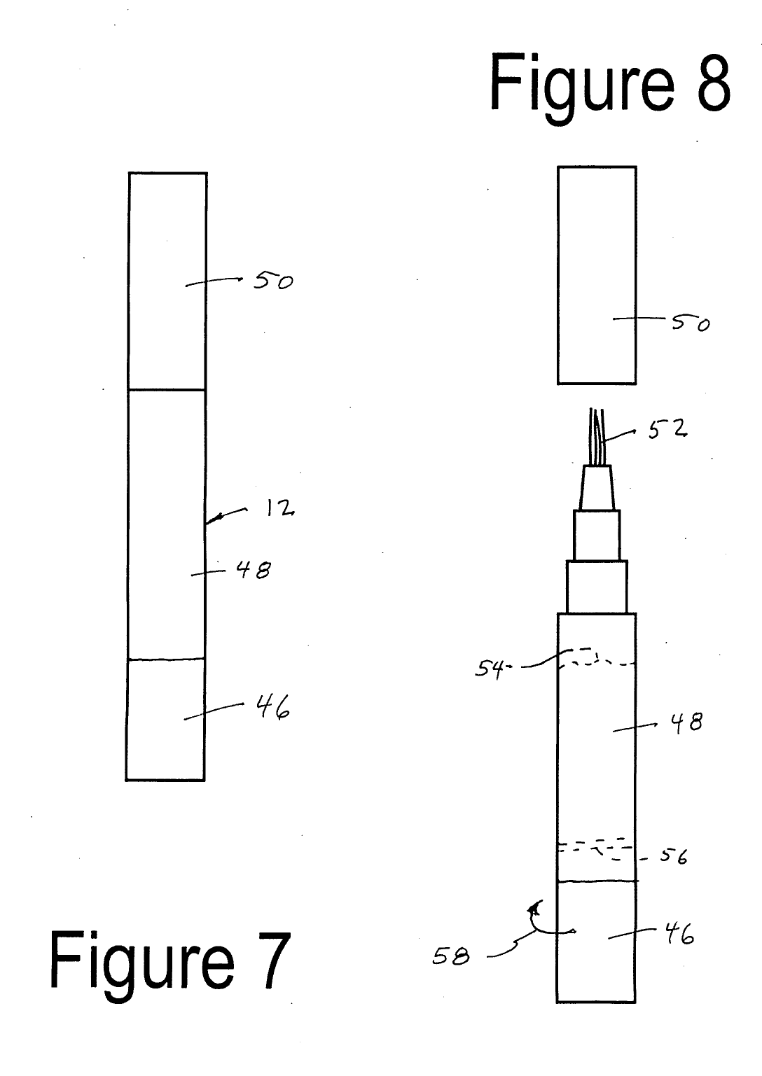

Referring to FIG. 7, applicator 12 generally comprises a crank portion 46, a body portion 48 and a cap 50.

As shown in FIG. 8, cap 50 may be removed exposing a brush 52 which is fed with, for example, paste-like tooth whitening material 54 which is advanced into brush 52 by a screw applicator plunger 56 which is operated by rotation of crank portion 46 in the direction of arrow 58.

During use, wedding favor 10 is distributed to members of the wedding party and, optionally, guests. Applicator 12 is, for example, a bright silvery color, white, pearlescent silver, other pearlescent color, other bright color or shiny black in attractive format which will encourage users to use the same publicly. In accordance with the invention it is contemplated that the applicator and ribbons may be colored to match the color of the bridal party. Alternatively, white ribbons may be used, as may be silver ribbons.

Further in accordance with the invention public use of the applicator 12 by some members of the party causes other individuals at the wedding or event to use the same. This increases the effectiveness of tooth whitening for the group as a whole.

In accordance with the invention, the inventive applicator is used by rotating crank portion 58 advancing whitening material 54 or any other suitable oral hygiene material into brush 52. Brush 52 may then be brushed against the teeth, applying the material to the teeth or, in the case of a gum treatment material to the gums or the teeth and gums of the user.

As alluded to above, the normal hesitancy of people to try new products and to benefit from them is committee ameliorated by the influence of the individuals around them trying the inventive wedding favor.

The inventive wedding favor also has the advantage that no package is necessary to house the various parts of the favor.

It is believed that the inventive device is the kind of favor wedding guests take home with them and remember and talk about as well, thus promoting the oral hygiene objectives of the same.

While an illustrative embodiment of the invention has been described, other modifications will be obvious to those of ordinary skill in the art and which are within the spirit and scope of the invention. The invention is limited only by the appended claims.

What is claimed:

1. A method of encouraging oral hygiene habits, comprising:
(a) putting on an affair;
(b) distributing at the affair a favor made by a method comprising joining together;
(i) an oral hygiene device, and
(ii) a card describing its use.

2. A method as in claim 1 wherein the oral hygiene device is shiny silver in color.

3. A method as in claim 1 wherein the affair is a wedding

4. A method as in claim 1 wherein said making of said favor further comprises joining a photograph to said card.

5. A method as in claim 4, wherein said joining of said photograph to said card is done using a glue.

6. A method as in claim 5 wherein said glue is silicone adhesive.

7. In a method as in claim 6 wherein said joining together is done with ribbon passing through a hole in said card.

8. In a method as in claim 1 wherein said joining together is done with ribbon passing through a hole in said card.

9. A party favor, comprising an instruction card, said instruction card defining a hole; a ribbon; an applicator; a photograph secured to said instruction card by said ribbon, said ribbon passing through said hole and being tied around said applicator.

10. A party favor as in claim 9, wherein said applicator contains a tooth whitening material.

11. A method of encouraging oral hygiene habits, comprising:
(a) putting on an affair;
(b) distributing at the affair a favor made by a method comprising joining together;
(i) an oral hygiene device, and
(ii) a card describing its use.
wherein the oral hygiene device is shiny silver in color, wherein the affair is a wedding, said making of said favor further comprises joining a photograph to said card, said joining of said photograph to said card is done using a glue, and said joining together is done with ribbon passing through a hole in said card.
